SEQUENCE LISTING (10 sequences)

SEQ ID NO: 1 - DNA, 26 bp, Artificial
  primer
tctagaaagacatggcctggaaacag

SEQ ID NO: 2 - DNA, 20 bp, Artificial
  primer
cccaacatggatcaggaact

SEQ ID NO: 3 - DNA, 45 bp, Artificial
  primer
gtgaagcaggctccaggaaagtgtttaaagtggatgggctggata

SEQ ID NO: 4 - DNA, 45 bp, Artificial
  primer
tatccagcccatccactttaaacactttcctggagcctgcttcac

SEQ ID NO: 5 - DNA, 30 bp, Artificial
  primer
ggatcccagatccagttggtgcagtctgga

SEQ ID NO: 6 - DNA, 36 bp, Artificial
  primer
tccggaagcttttgaggagacggtgactgaggttcc

SEQ ID NO: 7 - DNA, 42 bp, Artificial
  primer
taagaaggagatatacatatgcaaattgttctcacccagtct

SEQ ID NO: 8 - DNA, 57 bp, Artificial
  primer
gctttgttagcagccgaattcctattttatttccaactttgtcccacagccgaacgt

SEQ ID NO: 9 - DNA, 318 bp, Mus musculus
  CDS (1)..(318)
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccacgttcggctcggggacaaagttggaaataaaa

SEQ ID NO: 10 - DNA, 345 bp, Mus musculus
  CDS (1)..(345)
cagatccagttggtgcagtctggacctgaactgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatactgatgacttcaagggacggtttgccttctctttggaaacctctgccagcactgcctatttgcagatcaacaacctcaaaaatgaggacatggctacatatttctgtgcaagaaagggtgctatggactactggggtcaaggaacctcagtcaccgtctcctca

(19) United States
(12) Patent Application Publication
(10) Pub. No.: US 2014/0242073 A1
(43) Pub. Date: Aug. 28, 2014

(54) CARTILAGE/BONE DESTRUCTION SUPPRESSOR

(71) Applicants: Takami Matsuyama, Kagoshima (JP); Taku Nagai, Kagoshima (JP)

(72) Inventors: Takami Matsuyama, Kagoshima (JP); Taku Nagai, Kagoshima (JP)

(73) Assignee: KAGOSHIMA UNIVERSITY, Kagoshima-shi, Kagoshima (JP)

(21) Appl. No.: 14/240,267

(22) PCT Filed: Aug. 10, 2012
(86) PCT No.: PCT/JP2012/070872

§ 371 Date: Feb. 21, 2014

(30) Foreign Application Priority Data
Aug. 22, 2011 (JP) 2011-180899

(51) Int. Cl. C07K 16/28; A61K 38/16; A61K 39/395; C07K 14/21

(52) U.S. Cl. CPC C07K 16/28; C07K 14/21; A61K 38/164; A61K 39/3955; A61K 2039/505
USPC 424/134.1; 424/143.1

(57) ABSTRACT

An object of the present invention is to provide a cartilage/bone destruction suppressor which can suppress the destruction of cartilage or bone seen in rheumatoid arthritis, osteoarthritis, bone metastasis of malignant tumor, or the like. The present invention relates to a cartilage or bone destruction suppressor comprising an antibody against folate receptor β or a complex of the antibody and a biologically or chemically active substance.

### TECHNICAL FIELD

The present invention relates to a cartilage/bone destruction suppressor using an antibody or a complex thereof.

### BACKGROUND ART

Osteoarthritis (OA) is a disease producing the collapse of the joint cartilage surface and, concomitantly therewith, the proliferation of new cartilage at the periphery of the joint and the deformity and the failure of adaptability of the joint, due to aging or mechanical stress, and further progressing into the inflammation of the joint synovium. On the other hand, in rheumatoid arthritis (RA) as typical arthritis, the infiltration of inflammatory cells, due to immune abnormality or infection, occurs in the synovium; vascularization is further accompanied by the acceleration of proliferation of synovial fibroblasts to form inflammatory synovial granulation tissue; the destruction of bone and cartilage advances; and irreversible impairment is produced in the joint. For this reason, rheumatoid arthritis (RA) is an autoimmune disease called an inflammatory disease, whereas osteoarthritis (OA) is called a non-inflammatory disease. Thus, therapeutic agents used for the treatment of rheumatoid arthritis are generally considered to have no therapeutic effect for osteoarthritis.

Various pharmaceutical compositions have conventionally been developed for the purpose of treating rheumatoid arthritis (RA). One of them is an anti-Fas antibody (Patent Document 1). However, it is reported that although the anti-Fas antibody has the effect of inducing apoptosis on synovial cells collected from a rheumatoid arthritis (RA) patient, it does not have the effect of inducing apoptosis on synovial cells collected from an osteoarthritis (OA) patient (Non-Patent Document 1).

Adrenocortical hormones or hyaluronic acid preparations are now used for intra-articular administration in rheumatoid arthritis and osteoarthritis; however, their effects are temporary, and although they are effective against inflammation, it has not yet been determined whether they have a cartilage/bone destruction-suppressing effect (Non-Patent Documents 2 and 3). The systemic administration of various biological products (an anti-TNFα antibody and the like) is shown to have the effect of suppressing inflammation and cartilage and bone destruction in RA; however, some types of the arthritis are resistant to treatment even by such systemic administration (Non-Patent Document 4). When the biological products are each further intra-articularly administered in the rheumatoid arthritis (RA) resistant to the systemic administration of these biological products, it has not yet been determined whether they exert a cartilage/bone destruction-suppressing effect (Non-Patent Document 5). It is easily deduced from previous studies of the present inventors that an anti-folate receptor β (FR-β) immunotoxin suppresses inflammation in a disease characterized by the pathological condition mainly caused by FR-β-expressing macrophages; however, the suppression of inflammation does not always suppress the destruction of cartilage and bone as seen in the administration of an adrenocortical hormone or hyaluronic acid or as described in Non-Patent Document 6. In fact, the destruction of cartilage/bone in RA is considered to be a result of the complex entanglement of osteoclasts differentiating from macrophages, cytokines, such as IL-1 and TNF-α, produced by macrophages, and metalloprotease and the like produced by macrophages and fibroblasts (Non-Patent Documents 7 to 9).

Patent Document 2 states that the immunotoxin formed by the binding of an IgG type antibody against folate receptor β to a toxin (Pseudomonas exotoxin) induces cell death (apoptosis) in synovial cells of rheumatoid arthritis patients; however, in the document, the effect of suppressing the destruction of cartilage/bone is not confirmed and osteoarthritis (OA) is not addressed.

### CITATION LIST

### Patent Document

[Patent Document 1] JP 2004-59582 A
[Patent Document 2] WO2005/103250

### Non-Patent Document

[Non-Patent Document 1] NAKAJIMA et al., APOPTOSIS AND FUNCTIONAL FAS ANTIGEN IN RHEUMATOID ARTHRITIS SYNOVICYTES, ARTHRITIS & RHEUMATISM, 38(4), 1995, p485-p491.
[Non-Patent Document 2] Habib G S, Saliba W, Nashashibi M. Local effects of intra-articular corticosteroids. Clin Rheumatol. 2010 April; 29(4): 347-56.
[Non-Patent Document 3] Saito S, Kotake S. Is there evidence in support of the use of intra-articular hyaluronate in treating rheumatoid arthritis of the knee? A meta-analysis of the published literature. Mod Rheumatol. 2009; 19(5): 493-501.
[Non-Patent Document 4] Romas E, Gillespie M T. Inflammation-induced bone loss: can it be prevented? Rheum Dis Clin North Am. 2006 November; 32(4): 759-73.
[Non-Patent Document 5] Fisher B A, Keat A. Should we be using intraarticular tumor necrosis factor blockade in inflammatory monoarthritis? J. Rheumatol. 2006 October; 33(10): 1934-5.
[Non-Patent Document 6] van den Berg W B. Uncoupling of inflammatory and destructive mechanisms in arthritis. Semin Arthritis Rheum. 2001 April; 30(5 Suppl 2): 7-16
[Non-Patent Document 7] Udagawa N, Kotake S, Kamatani N, Takahashi N, Suda T. The molecular mechanism of osteoclastogenesis in rheumatoid arthritis. Arthritis Res. 2002; 4(5): 281-9.
[Non-Patent Document 8] Catrina A I, Lampa J, Ernestam S, of Klint E, Bratt J, Klareskog L, Ulfgren A K. Anti-tumour necrosis factor (TNF)-alpha therapy (etanercept) down-regulates serum matrix metalloproteinase (MMP)-3 and MMP-1 in rheumatoid arthritis. Rheumatology (Oxford). 2002 May; 41(5): 484-9.
[Non-Patent Document 9] Schiff M H. Role of interleukin 1 and interleukin 1 receptor antagonist in the mediation of rheumatoid arthritis. Ann Rheum Dis. 2000 November; 59 Suppl 1: i103-8.

### SUMMARY OF INVENTION

### Technical Problem

An object of the present invention is to provide a cartilage/bone destruction suppressor which can suppress the destruction of cartilage or bone seen in rheumatoid arthritis, osteoarthritis, bone metastasis of malignant tumor, or the like.

### Solution to Problem

The subject matter of the present invention is as follows.

(1) A cartilage or bone destruction suppressor comprising an antibody against folate receptor β or a complex of the antibody and a biologically or chemically active substance.

(2) The cartilage or bone destruction suppressor according to (1) above, wherein the antibody against folate receptor β is in the form of a single-chain or a double-chain.

(3) The cartilage or bone destruction suppressor according to (1) or (2) above, wherein the biologically or chemically active substance is at least one selected from toxins, enzymes, cytokines, isotopes, and chemotherapeutic agents.

(4) The cartilage or bone destruction suppressor according to any of (1) to (3) above for treating a disease characterized by the destruction of cartilage or bone caused by folate receptor β-expressing macrophages.

(5) The cartilage or bone destruction suppressor according to any of (1) to (3) above for suppressing the destruction of cartilage or bone due to rheumatoid arthritis, osteoarthritis, or bone metastasis of malignant tumor.

### Advantageous Effects of Invention

According to the present invention, a cartilage/bone destruction suppressor can be provided which can suppress the destruction of cartilage or bone seen in rheumatoid arthritis, osteoarthritis, bone metastasis of malignant tumor, or the like.

### BRIEF DESCRIPTION OF DRAWINGS

FIG. 1 is a series of graphs showing the binding capacity of a mouse anti-rat FR-β monoclonal antibody, 4A67, to FR-β-expressing cells.

FIG. 2 is a diagram showing the VL gene sequence of a mouse anti-rat FR-β antibody, 4A67, and the deduced amino acid sequence thereof.

FIG. 3 is a diagram showing the VH gene sequence of a mouse anti-rat FR-β antibody, 4A67, and the deduced amino acid sequence thereof.

FIG. 4 is a graph showing the cell proliferation-suppressing effect (the apoptosis-inducing capacity) of mouse anti-rat FR-β immunotoxin on FR-β-expressing B300-19 cells.

FIG. 5 is a graph showing the joint swelling-suppressing effect of the administration of an immunotoxin in methylated bovine serum albumin-induced rat arthritis.

FIG. 6 is a series of drawings showing the results of pathological analysis in the administration of an immunotoxin in methylated bovine serum albumin-induced rat arthritis.

FIG. 7 is a pair of photographs showing FR-β-expressing cells in a bone destruction site in rheumatoid arthritis.

FIG. 8 is a series of photographs showing FR-β-expressing cells in the osteoarthritic synovium.

FIG. 9 is a pair of photographs showing FR-β-expressing cells in a site of bone metastasis of liver cancer.

FIG. 10 is a pair of drawings showing the schematic and results of a method for detecting a rat serum antibody against the toxin of an anti-FR-β immunotoxin.

### DESCRIPTION OF EMBODIMENTS

An antibody against folate receptor β (FR-β) (an anti-FR-β antibody) may be used as an active ingredient of the cartilage/bone destruction suppressor of the present invention; however, it is preferable to use a complex of the anti-FR-β antibody and a biologically or chemically active substance.

Examples of the biologically or chemically active substance include toxins, enzymes, cytokines, isotopes, and chemotherapeutic agents, preferably toxins.

In a preferable aspect of the present invention, the DNAs of the antigen-binding site of the H-chain or L-chain of the antibody and the toxin can be bound to each other by genetic engineering to produce a protein in Escherichia coli to prepare a recombinant single-chain immunotoxin or a recombinant double-chain immunotoxin. The recombinant immunotoxin easily enters cells because of its low molecular weight, and yet has the advantage of being capable of being purified in large amounts compared to that when the bound substance of the antibody and the toxin is chemically prepared.

It is known that a chimeric antibody is low in the production of an antibody against its mouse antibody portion in humans and is useful for clinical administration. In addition, it has been described that a humanized antibody in which CDR1, CDR2, and CDR3 of a human immunoglobulin are replaced with CDR1, CDR2, and CDR3 of the mouse Fab portion produces a small amount of antibodies against the mouse antibody portion and is useful in clinical administration.

In addition, a completely humanized antibody obtained from a human immunoglobulin Fab phage display library is known to be low in the production of an antibody against the administered antibody portion and useful for clinical administration.

As used herein, the term “antibody” refers to a polyclonal antibody, a monoclonal antibody, an antibody adapted for human body, a single-chain antibody, and a fragment thereof such as a Fab fragment, a F(ab′)₂ fragment, or a Fv fragment, and other fragments maintaining the antigen-binding capacity of the parent antibody.

As used herein, the term “monoclonal antibody” means an antibody group constituting a single antibody population. This term is not limited as regards to the species and origin of the antibody and also not intended to be limited by a method for producing the antibody. The term encompasses an intact immunoglobulin as well as a Fab fragment, a F(ab′)₂ fragment, a Fv fragment, and other fragments maintaining the antigen-binding capacity of the antibody. Monoclonal antibodies of mammals and birds may also be used in the present invention.

As used herein, the term single-chain antibody shall refer to an antibody prepared by determining the binding regions (of both H- and L-chains) of an antibody having a binding capacity and imparting such a binding site that the binding capacity is maintained. This results in the formation of a thoroughly simplified antibody essentially having only a variable region site necessary for binding to an antigen. As used herein, the term “double-chain antibody” shall refer to an antibody prepared by determining the binding region (of both H- and L-chains) of an antibody having a binding capacity and S—S binding the H-chain or the L-chain and the L-chain or the H-chain. This results in the formation of a thoroughly simplified antibody essentially having only a variable region site necessary for binding to an antigen.

For the purpose of the present invention, the immunotoxin (IT) shall refer to a chimeric molecule in which a ligand binding to cells has been bound to a toxin or a subunit thereof. The toxin portion of the immunotoxin is derived from any of various sources such as plants and bacteria; human-derived toxins or synthetic toxins (drugs) may be similarly used.

Preferably, the toxin portion is derived from a plant toxin such as a type 1 or 2 ribosome-inactivating protein (RIP). The type 2 ribosome-inactivating protein contains, for example, ricin. The type 1 RIP is particularly advantageous to constructing an immunotoxin by the present invention.

Examples of the toxin include Pseudomonas exotoxin, ricin A chain, deglycosylated ricin A chain, ribosome-inactivating protein, α-sarcin, gelonin, bryodin, momordin, saporin, bouganin, aspergillin, restrictocin, ribonuclease, epipodophyllotoxin, and diphtheria toxin.

The ligand portion of IT generally refers to a monoclonal antibody binding to selected target cells. The toxin portion of IT used in Examples of the present specification is Pseudomonas exotoxin (PE) as a bacteria-derived toxin. Specifically, it has an ADP-ribosylating activity and a capability of translocation through a cell membrane. More specifically, PE becomes an active form by the cleavage between positions 279 and 280 of the amino acid sequence, and can be prepared by gene-introducing, into Escherichia coli, an expression plasmid containing DNA encoding PE devoid of the receptor-binding domain Ia of the natural toxin.

According to the present invention, the PE-binding recombinant immunotoxin lacks an Ia domain for binding to the cell surface, starts with position 280 of the amino acid sequence, and has KDEL and/or REDLK added to the C-terminal site to increase a cytotoxic capability. Specifically, the absence of a binding activity to cells in the toxin markedly decreases non-specific toxicity. More specifically, the gene-modified PE has low toxicity to human or animal cells in vitro and has low toxicity to the liver when administered in vivo, compared to the unmodified PE.

In addition, for the purpose of the present invention, the recombinant single-chain immunotoxin refers to a protein prepared by binding DNAs of the antigen-binding site of H- or L-chain of the antibody and a toxin to each other by genetic engineering and producing a protein in Escherichia coli. Specifically, the recombinant single-chain immunotoxin generally refers to one containing an intervening sequence of about 15 translated amino acids between H- and L-chains. (Reiter et al. Recombinant Fv immunotoxins and Fv fragments as novel agents for cancer therapy and diagnosis. Trends Biotechnol. 1998 December; 16(12): 513-20)

For the purpose of the present invention, the recombinant double-chain immunotoxin refers to one obtained by binding DNA of the antigen-binding site of the H- or L-chain to toxin DNA by genetic engineering, preparing a protein in Escherichia coli, separately preparing a protein from DNA of the antigen-binding site of the L- or H-chain, and binding these proteins to each other by S—S binding. (Brinkmann et al. A recombinant immunotoxin containing a disulfide-stabilized Fv fragment. Proc Natl Acad Sci USA. 1993; 90(16): 7538-42)

For the purpose of the present invention, the chimeric antibody refers to one obtained by binding DNA of the antigen-binding site (Fab portion) of mouse immunoglobulin to DNA of the Fc portion of human-derived immunoglobulin by genetic engineering, followed by production in Escherichia coli. (Smith et al. Rituximab (monoclonal anti-CD20 antibody): mechanisms of action and resistance. Oncogene. 2003; 22(47): 7359-68)

For the purpose of the present invention, the humanized antibody refers to an antibody in which CDR1, CDR2, and CDR3 of a human immunoglobulin are replaced with CDR1, CDR2, and CDR3 of a mouse Fab portion (Kipriyanov. Generation and production of engineered antibodies. Mol. Biotechnol. 2004; 26(1): 39-60), and a completely humanized antibody obtained from a human immunoglobulin Fab phage display library. (Feng Y et al. A folate receptor beta-specific human monoclonal antibody recognizes activated macrophage of rheumatoid patients and mediates antibody-dependent cell-mediated cytotoxicity. Arthritis Res Ther. 2011; 13(2): R59)

For the purpose of the present invention, the liposome refers to one in which a drug is wrapped with a lipid membrane, as a drug delivery system. Specifically, it refers to one containing an antibody specifically binding to cells in addition to a drug for the specific delivery of the drug to cells. (Gabizon et al. Targeting folate receptor with folate linked to extremities of poly(ethylene glycol)-grafted liposomes: in vitro studies. Bioconjug Chem. 1999; 10(2): 289-98)

Examples of the biologically or chemically active enzyme according to the present invention include urokinase, plasmin, plasminogen, staphylokinase, and thrombin which act on the coagulation system, and metalloprotease, collagenase, gelatinase, and stromelysin as proteases.

Examples of the cytokine according to the present invention include interferon, TGF-β, and TNF-α which have an anti-tumor effect, endostatin which has a vascularization-suppressing effect, and IL-1 receptor antagonists, IL-4, IL-10, IL-19, IL-20, IL-22, IL-24, IL-26, IL-28, and IL-29 which have an anti-inflammatory effect.

Examples of the isotope according to the present invention include gallium 67, gallium 68, indium 111, indium 113, iodine 123, iodine 125, iodine 131, technetium 99, yttrium 90, rubidium 97, and rubidium 103.

For the purpose of the present invention, the chemotherapeutic agent refers to a molecule having a cytotoxic capability. Specific examples thereof include cytosine arabinoside, fluorouracil, methotrexate, aminopterin, anthracycline, mitomycin, demecolcine, etoposide, and mithramycin as metabolic antagonists, chlorambucil, melphalan, and endoxan as alkylating agents, daunorubicin, doxorubicin, and adriamycin as DNA synthesis inhibitors, and vinca alkaloids such as colchicine, taxane, vinblastine, and vincristine as tubulin polymerization inhibitors.

For the purpose of the present invention, the folate receptor β (FR-β) refers to a molecule which is a surface antigen expressed in activated macrophages and acute myelocytic leukemia and is involved in the intracellular delivery of folic acid.

The antibody used in the present invention is preferably an FR-β monoclonal antibody. It may be of IgM type, IgG type, or the like. Examples of the FR-β monoclonal antibody used in the present invention include one produced from clonal cells obtained by immunizing a mouse with FR-β-expressing B300-19 cells and then fusing splenic cells of the mouse to mouse myeloma cells.

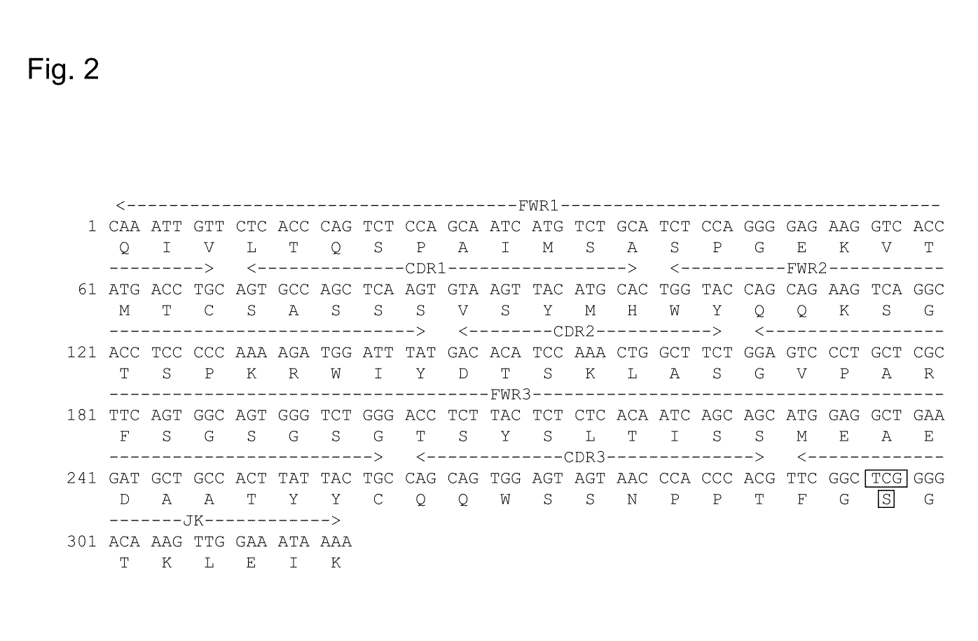

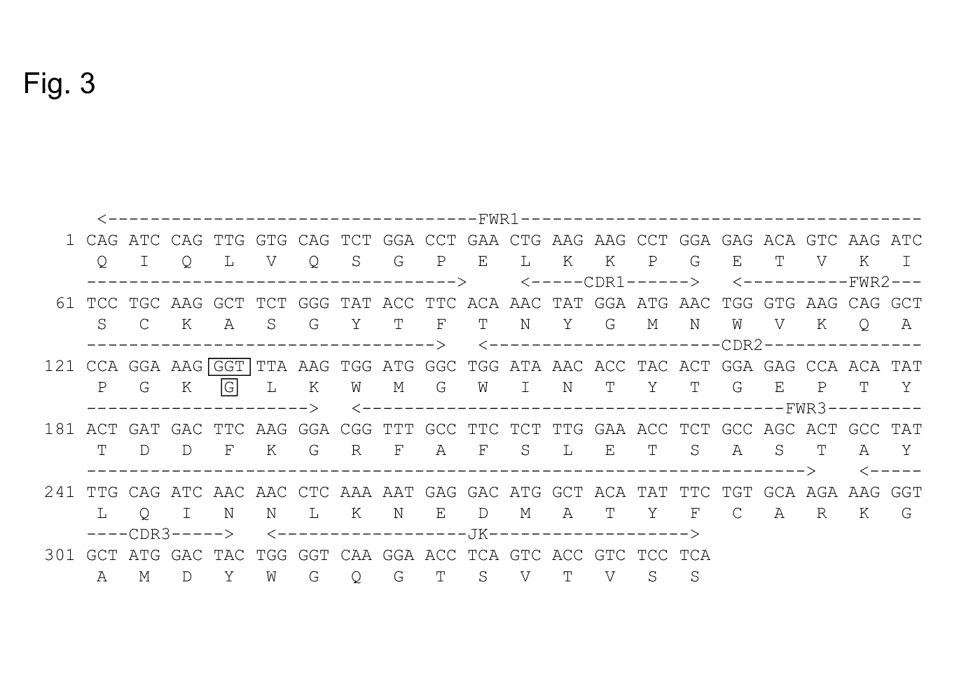

FIG. 2 and SEQ ID NO: 9 show the gene (VL gene) and deduced amino acid sequence of the L-chain of the mouse anti-rat FR-β antibody, 4A67, used in Examples of the present specification and FIG. 3 and SEQ ID NO: 10 show the gene (VH gene) and deduced amino acid sequence of the H-chain thereof.

The antibody derived from clone 94b or clone 36 cells producing an FR-β monoclonal antibody described in WO2005/103250 (Patent Document 2) can also be used in the present invention; however, according to the present invention, preferred is a recombinant FR-β antibody immunotoxin using the antibody derived from clone 94b cells.

The nucleotide sequence of the gene of the H-chain of clone 36 cells is described in SEQ ID NO: 1 of the Sequence Listing of WO2005/103250 (Patent Document 2), and the nucleotide sequence of the gene of the H-chain of clone 94b cells is described in SEQ ID NO: 3 of the Sequence Listing of WO2005/103250 (Patent Document 2).

The FR-β monoclonal antibodies described in WO2005/103250 (Patent Document 2) include the genes of the H- and L-chains of clone 94b or clone 36 cells producing an FR-β monoclonal antibody and the proteins encoded by the genes.

Variants having biological activities substantially equivalent to those of these genes or the protein can also be used in the present invention. Humanized FR-β monoclonal antibodies obtained by chimerizing the genes of the H-chains of these clone cells producing an FR-β monoclonal antibody and the genes of the L-chains thereof can also be used in the present invention.

Active ingredients of the present invention also include a recombinant FR-β antibody immunotoxin using the gene of the H-chain of each clone cells producing an FR-β monoclonal antibody and the gene of the L-chain thereof.

The nucleotide sequence of the gene of the H-chain of clone 36 cells is described in SEQ ID NO: 1 of the Sequence Listing of WO2005/103250 (Patent Document 2), and the nucleotide sequence of the gene of the H-chain of clone 94b cells is described in SEQ ID NO: 3 of the Sequence Listing of WO2005/103250 (Patent Document 2).

In the present invention can also be used a gene in which a part, e.g., 20 or less, preferably 10 or less, more preferably 5 or less nucleotides, of each of the above nucleotide sequences are deleted, substituted, or added, a gene having 90% or more, preferably 95% or more, more preferably 99% or more homology to each of the above nucleotide sequences, and a gene capable of hybridizing to the gene having the above nucleotide sequence under stringent conditions, provided that each of them encodes a protein having biological activities substantially equivalent to those of the H- or L-chain of corresponding clone cells producing an FR-β monoclonal antibody.

According to genetic engineering techniques, a particular site of fundamental DNA can be subjected to artificial mutation without changing the basic characteristics of the DNA or so that the characteristics are improved.

In the present invention can also be used a protein in which a part, e.g., 20 or less, preferably 10 or less, more preferably 5 or less amino acids, of each of the amino acid sequences encoded by the above nucleotide sequences are deleted, substituted, or added, and a protein having 95% or more, preferably 97% or more, more preferably 99% or more homology to each of the amino acid sequences encoded by the above nucleotide sequences, provided that each of them has biological activities substantially equivalent to those of the H- or L-chain of corresponding clone cells producing an FR-β monoclonal antibody.

As used herein, “substantially equivalent” means being substantially the same in activities of each protein, for example, physiological or biological activities such as specific binding to the FR-β antigen. The meaning of the term may include the case of having activities of substantially the same quality; the “activities of substantially the same quality” means having activity properties of the same quality, such as specific binding to the FR-β antigen, and, for example, means having the same physiological, pharmacological, or biological quality. The quantitative degrees of the activities are preferably the same; however, quantitative elements may be different.

According to the present specification, the “stringent” conditions of hybridization can be properly selected by those skilled in the art; however, as a specific example, the hybridization can be performed using the following operations. A membrane to which a DNA or RNA molecule to be tested is transferred is hybridized to a labeled probe in a suitable hybridization buffer. The composition of the hybridization buffer comprises, for example, 5×SSC, 0.1% by weight of N-lauroyl sarcosine, 0.02% by weight of SDS, 2% by weight of a blocking reagent for nucleic acid hybridization, and 50% formamide. The blocking reagent for nucleic acid hybridization may be, as an example, a commercial blocking reagent for nucleic acid hybridization dissolved to 10% in a buffer (pH 7.5) comprising of 0.1 M maleic acid and 0.15 M sodium chloride. 20×SSC is a solution of 3 M sodium chloride and 0.3 M citric acid, and SSC is more preferably used in a concentration of 3 to 6×SSC, still more preferably 4 to 5×SSC.

The hybridization temperature is in the range of 40 to 80° C., more preferably 50 to 70° C., still more preferably 55 to 65° C. Incubation is performed for several hours to overnight, followed by washing with a washing buffer. The washing temperature is preferably room temperature, more preferably temperature during the hybridization. The composition of the washing buffer is 6×SSC+a 0.1% by weight SDS solution, more preferably 4×SSC+a 0.1% by weight SDS solution, still more preferably 2×SSC+a 0.1% by weight SDS solution, yet more preferably 1×SSC+a 0.1% by weight SDS solution, most preferably 0.1×SSC+a 0.1% by weight SDS solution. The membrane is washed with the washing buffer and the DNA or RNA molecule hybridized to the probe can be identified using the label used in the probe.

An example of a preferred embodiment of the present invention will be described below.

### [Preparation of FR-β-Expressing Cell]

FR-β-expressing B300-19 cells are prepared by the following method. First, FR-β gene is incorporated into pEF-BOS vector. The vector is not limited to pEF-BOS vector, and may be any mammalian expression vector. Next, the FR-β gene is transfected into mouse B-300-19 cells by a lipofectamine method. The gene transfer method may be an electroporation method. The cell line may be any cell line derived from a Balb/C mouse.

These cells are immunized to prepare an IgM type or IgG type FR-β monoclonal antibody of a low molecular weight, having a high affinity to the FR-β antigen, by a cell fusion method. The antibody and a toxin (toxin molecule) are chemically bound to each other by any of various well-known chemical methods, for example, using a cross-linker having different divalent binding groups, such as SPDP, carbodiimide, and glutaraldehyde. The production of various immunotoxins is well known in the art, and is described, for example, in Monoclonal Antibody-Toxin Conjugates: Aiming the Magic Bullet, Thorpe et al. Monoclonal Antibodies in Clinical Medicine, Academic Press, pp. 168-190 (1982) and Waldman, Science, 252: 11657 (1991). These two literatures are made part of the present specification by reference.

### [Preparation of FR-β Antibody Immunotoxin]

The antibody is bound to a toxin, preferably Pseudomonas exotoxin (PE), using succinimidyltrans-4-(maleimidylmethyl)cyclohexane 1-carboxylate (SMCC) according to the method of Haasan et al. (Haasan et al. Anti-tumor activity of K1-LysPE38QQR, an immunotoxin targeting mesothelin, a cell-surface antigen overexpressed in ovarian cancer and malignant mesothelioma. J. Immunother. 2000 J; 23(4): 473-9) to prepare an immunotoxin.

The antibody can be fused to the toxin by a recombination technique as in the step of preparing the single-chain antibody-toxin fusion protein. A gene encoding the ligand and a toxin gene are each cloned in cDNA using a well-known cloning method; they are bound directly or apart from each other through a small peptide linker. For example, reference is made to Sambrook et al, Molecular Cloning: A Laboratory Manual, Cold Spring Harbor Laboratory (1989).

### [Action Effect of Anti-FR-β Immunotoxin]

The anti-FR-β immunotoxin is also effective in suppressing cartilage destruction in osteoarthritis in which macrophages are considered to cause cartilage and bone destruction as in rheumatoid arthritis and bone destruction in bone metastasis of brain tumor, malignant melanoma, pancreas cancer, breast cancer, prostate cancer, myeloma, large bowel cancer, kidney cancer, stomach cancer, uterine cancer, and thyroid cancer.

### [Dose and Method of Administration of FR-β Antibody Immunotoxin]

The immunotoxin is administered at a concentration effective in suppressing cartilage and bone destruction in rheumatoid arthritis, osteoarthritis, and the like and bone destruction in bone metastasis of malignant tumors. To accomplish this purpose, the immunotoxin is formulated using various acceptable excipients known in the art. Typically, the immunotoxin is intravenously or intra-articularly administered by injection. The composition of the present invention is formulated in a unit dosage form for injection, such as a solution, a suspension, or an emulsion by mixing it with pharmaceutically acceptable parenteral excipients. Such excipients are essentially nontoxic, and nontherapeutic. Examples of such excipients include saline, Ringer's solution, dextrose solution, and Hank's solution. Nonaqueous excipients such as fixed oil and ethyl oleate may be used. A preferable excipient is 5% dextrose in saline. The excipient may contain small amounts of additives such as substances enhancing isotonicity and chemical stability, including buffer and preservative.

The dose and the dosage form will be dependent on individuals. The composition is generally administered so that the immunotoxin is most preferably administered at a dose of 0.1 to 2 μg/kg. It is preferably administered by bolus dosing. Continuous infusion may also be used. According to a particular case, the necessary “therapeutic effective dose” of the immunotoxin should be decided in the form of a dose sufficient for treating a patient requiring such treatment or for at least partially pausing the appropriate disease and its complications. The dose effective for such use will be dependent on the severity of the disease and the systemic health of the patient. Single administration or multiple administration is required depending on the dose required and administration frequency withstood by a patient.

The dosage form is preferably for intra-articular administration for osteoarthritis and rheumatoid arthritis and is for systemic administration or local administration for bone metastasis of malignant tumor.

The present specification encompasses the contents of the specification and/or drawings of Japanese Patent Application No. 2011-180899 on which the priority of the present application is based.

### EXAMPLES

### Example 1

### Preparation of Mouse Anti-Rat FR-β Monoclonal Antibody

### [Preparation of FR-β-Expressing Cell as Antigen]

Total RNA was extracted from the liver of Lewis rats using Trizol (GibcoBRL) and a cDNA synthesis kit (Invitrogen) according to the accompanying instruction manual, and cDNA was then synthesized using SuperScript plasmid System (Invitrogen) according to the accompanying instruction manual. The Lewis rat liver cDNA was added to Bioneer PCR premix (Bioneer Corporation); thereto were then added a sense primer (rat liver: tct aga aag aca tgg cct gga aac ag SEQ ID NO: 1) and an antisense primer (ccc aac atg gat cag gaa ct SEQ ID NO: 2) each adjusted to an amount of 10 picomoles; and a 30-cycle PCR was performed at 94° C. for 20 seconds, 58° C. for 30 seconds, and 72° C. for 60 seconds, followed by reaction at 72° C. for 5 minutes to amplify the rat FR-β gene. PCR product of the amplified FR-β gene was ligated to pTAC-1 (Biodynamic Laboratory). Specifically, to 2.5 μl of the PCR product were added 1 μl of NaCl solution, 1.5 μl of sterile distilled water, and 1 μl of vector plasmid (PCR2.1-TOPO), which was then incubated at room temperature for 5 minutes; 2 μl thereof was added to Escherichia coli (TOP10F′), which was then reacted in ice for 30 minutes, heat-treated at 42° C. for 30 seconds, and allowed to stand in ice for 2 minutes; and 250 μl of SOC medium was added thereto, which was then cultured in a shaker at 37° C. for 1 hour. After the end of culture, the resultant was seeded on an LB medium and cultured at 37° C. overnight.

For the culture of Escherichia coli, white colonies collected from the plate were added to a liquid LB medium containing ampicillin (0.1 mg/ml) and cultured at 37° C. overnight. The plasmid was purified using a Qiagen plasmid purification kit (Qiagen). The incorporated FR-β gene was treated with a restriction enzyme, EcoRI and developed by agarose electrophoresis to confirm an FR-β gene product of about 0.8 kb (782 bp), and the site thereof was then cut out, followed by purifying an extract of the gene product using a Quiagen PCR purification kit (Quiagen). The resultant was mixed with a vector for mammalian cell expression, pEF-BOS (Mizushima et al. pEF-BOS, a powerful mammalian expression vector. Nucleic Acid Res. 1990; 18(17): 5322) which was treated with EcoRI in advance, followed by ligation using T4 ligase (Roche). The gene transfer of the ligation product into Escherichia coli (TOP10F′) and the confirmation of FR-β gene were carried out using the same technique as that described above.

After confirming the FR-β gene incorporated in pEF-BOS, it was transfected into mouse B300-19 cells. Specifically, the gene transfer was carried out by adding 1 μg of the FR-β vector mixed with 20 μl of lipofectamine (GibcoBRL) to each cells adjusted to 1×10⁵ cells in advance. Because the transfected B300-19 mouse cells and rat RBL2H3 cells acquire resistance to an antibiotic, G418, the transfected cells were subjected to selective culture in a medium containing G418 at a concentration of 1 mg/ml. The gene transfer of FR-β gene in the transfected cells was confirmed by a PCR method. Specifically, cDNA was synthesized with a cDNA synthesis kit (Invitrogen) using each cells adjusted to 1×10⁷ cells; thereto was then added a sense primer (rat liver: tct aga aag aca tgg cct gga aac ag SEQ ID NO: 1) and an antisense primer (ccc aac atg gat cag gaa ct SEQ ID NO: 2) each adjusted to an amount of 10 picomoles; and a 30-cycle PCA was performed at 94° C. for 20 seconds, 58° C. for 30 seconds, and 72° C. for 60 seconds, followed by reaction at 72° C. for 5 minutes to amplify the rat FR-β gene. After amplification, agarose electrophoresis was performed to confirm a band of 0.8 kb exhibited by the FR-β gene.

### [Preparation of Mouse Anti-Rat FR-β Monoclonal Antibody]

Rat FR-β-expressing mouse B300-19 cells were adjusted to 1×10⁷ cells, mixed with Freund's complete adjuvant, and immunized into three places on the tail section and into the peritoneal cavity of a Balb/C mouse. The immunization was repeated 2 to 4 times.

The monoclonal antibody was prepared according to the method of Kohler (Kohler & Milstein, Nature (1975) 256: 495-96). Specifically, the spleen or iliac lymph node was removed and dissociated into single cells. The dissociated cells were fused to myeloma-derived cells (NS-1) to prepare hybridomas, which were then cultured in HAT selection medium, and antibodies secreted into the culture supernatant were sorted by reactivity with the previous rat FR-β-expressing cells.

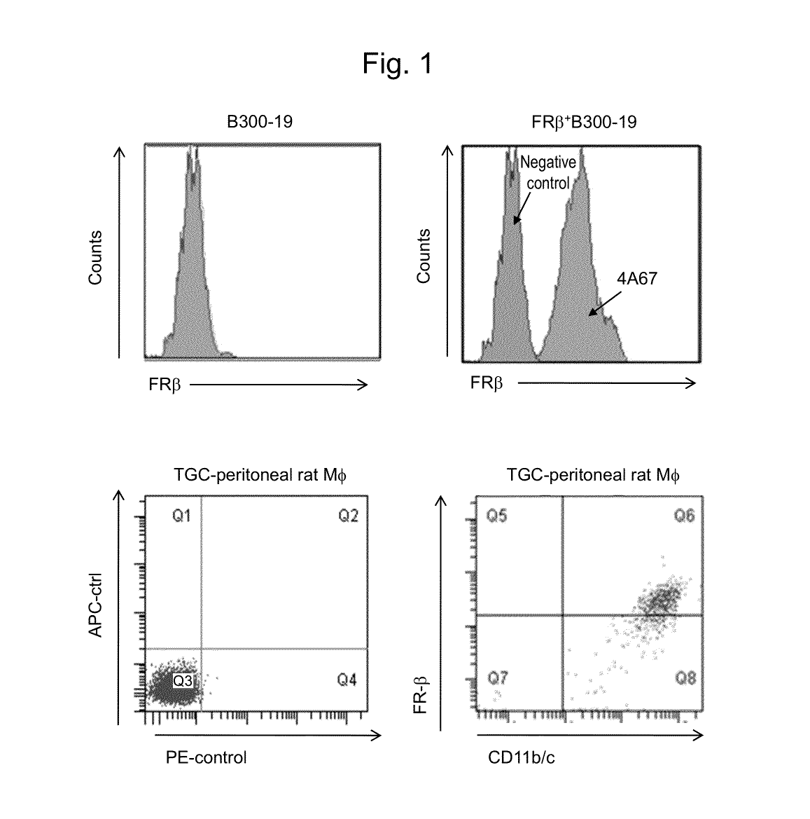

The resultant hybridomas were cloned by limiting dilution culture adjusted to 1 cell per well of a 96-well plate. The cloned cells were sorted by reactivity with FR-β-expressing cells. The isotype of the mouse monoclonal antibodies was determined using a mouse immunoglobulin isotyping ELISA kit (Pharmingen). As a result, the mouse anti-rat FR-β monoclonal antibodies obtained were IgM-type clone 4A67. The reactivity of these antibodies to the antigen was analyzed by flow cytometry. The results of the flow cytometry are shown in FIG. 1.

The top of FIG. 1 shows the results of reacting B300-19 cells (left) and FR-β-expressing B300-19 (right) each adjusted to 1×10⁵ cells with 4A67 antibody or a negative control antibody and further with an anti-mouse IgM antibody labeled with APC. After the end of reaction, stainability was measured using a flow cytometer. For the bottom, 3% thioglycolate was intraperitoneally administered to Lewis rats, and peritoneal macrophages were collected 4 days later. The negative control antibody or the 4A67 antibody was added to the macrophages adjusted to 1×10⁵ for the same reaction as above, followed by further adding an anti-CD11b/c antibody labeled with phycoerythrin or the negative control antibody labeled with phycoerythrin for reaction. After the reaction, stainability was measured using a flow cytometer. The left shows the negative control group and the right shows stainability by 4A67 and anti-CD11b/c.

It was demonstrated that the resultant antibody 4A67 reacts with rat FR-β-expressing B300-19 and thioglycolate-induced peritoneal macrophages.

### [Determination of Heavy Chain Gene Variable Region (VH) and Light Chain Gene Variable Region (VL) Gene of Mouse Anti-Rat FR-β Monoclonal Antibody]

Hybridoma clone 4A67 was adjusted to 1×10⁷ cells, and 4A67 for which cDNA was synthesized using a cDNA synthesis kit (Invitrogen) was determined for the genes of VH and VL by PCR using Ig-Prime Kit. PCR conditions were according to the accompanying instruction manual. Specifically, a 30-cycle PCR was performed at 94° C. for 60 seconds, 50° C. for 60 seconds, and 72° C. for 120 seconds, followed by reaction at 72° C. for 5 minutes to amplify the genes of VH and VL. The PCR products of the amplified VH and VL genes were ligated to plasmid PCR2.1-TOPO (Invitrogen), and transfected into Escherichia coli (TOP10F′). The plasmid was purified from the transfected Escherichia coli, and the genes of VH and VL of 4A67 were sequenced. The nucleotide sequences were subjected to PCR using BigDye Terminator V3.1 cycle sequencing kit (ABI), and the PCR products were analyzed using ABI 310 DNA sequencer.

FIG. 2 shows the VL gene sequence of the mouse anti-rat FR-β antibody 4A67 and the deduced amino acid sequence thereof. The 3rd amino acid of the JK portion mutated to cysteine is shown in a box. FIG. 3 shows the VH gene sequence of the mouse anti-rat FR-3 antibody 4A67 and the deduced amino acid sequence thereof. The mutated 9th amino acid of the FWR2 portion is shown in a box.

In FIGS. 2 and 3, FWR represents a framework region; CDR represents a hypervariable region (complementarity determining region); and JK represents a junction region.

### Example 2

### Preparation of Recombinant Immunotoxin

[Introduction of Cysteine Mutation into Immunoglobulin Heavy Chain Gene Variable Region (VH)]

Primers (sense: gtgaagcaggctccaggaaagTGTttaaagtggatgggctggata SEQ ID NO: 3; antisense: tatccagcccatccactttaaACActttcctggagcctgcttcac SEQ ID NO: 4) were prepared which were designed so that the 63rd amino acid glycine (nucleotide sequence: ggc) of the immunoglobulin heavy chain gene variable region (VH) of the mouse anti-rat FR-β monoclonal antibody 4A67 is mutated to cysteine (nucleotide sequence: tgt), and the plasmid pCR2.1-TOPO 4A67VH containing VH of 4A67 obtained in Example 1 was subjected to mutagenesis treatment using Quick change site-directed mutagenesis kit (Stratagene). The PCR reaction was carried out by subjecting the reaction solution to 12 continuous cycles of 95° C. for 30 seconds, 55° C. for 60 seconds, and 68° C. for 4 minutes.

DNA after the reaction was then transfected into Escherichia coli XL1-Blue, which was then subjected to selective culture in an LB medium containing 0.1 mg/ml of ampicillin. A plasmid in the selected transformant was purified using QIAprep spin Miniprep KIT (Qiagen). In addition, the nucleotide sequence thereof was determined using Big Dye Terminator v3.1 cycle sequencing kit (ABI) and ABI310 sequencer to confirm mutation to cysteine (nucleotide sequence: tgt).

[Insertion of Mutation-Introduced VH Gene into pRSETPE38 Vector]

The mutation-introduced 4A67VH gene was then inserted into a pRSET vector containing PE38 gene, pRSETPE38.

GGATCCcagatccagttggtgcagtctgga SEQ ID NO: 5 and tccggAAGCTTttgaggagacggtgactgaggttcc SEQ ID NO: 6 were designed as annealing primers for the 5′-terminal and 3′-terminal regions of the mutation-introduced 4A67, respectively. The annealing primers have restriction enzyme sites, BamHI and HindIII sites, respectively, and cloning between the sites enables the expression of a fused protein in which VH and PE genes are bound to each other.

The combination of these primers and pfu DNA polymerase (Stratagene) were used to subject the mutation-introduced plasmid pCR2.1-TOPO-4A67VH to PCR. The reaction comprises a 30-cycle PCR at 94° C. for 20 seconds, 55° C. for 30 seconds, and 72° C. for 60 seconds, followed by reaction at 72° C. for 5 minutes. Then, the PCR product was purified; the restriction enzymes BamHI (New England Biolabs) and HindIII (New England Biolabs) were added to the purified product for reaction; the resultant was subjected to electrophoretic development; and DNA of a desired size was recovered from the gel using QIAquick gel extraction kit (Qiagen). To the recovered DNA was added pRSETPE38 treated with the same restriction enzyme as for the restriction enzyme-treated mutation-introduced VH, and Ligation High (Toyobo) was further used to perform the ligation reaction between VH and pRSETPE38. After the end of the ligation reaction, the gene was introduced into the Escherichia coli TOP 10F′ (Invitrogen), and a transformant was selected in an LB medium containing 0.1 mg/ml ampicillin. The plasmid pRSET-VHPE in the selected transformant was purified using QIAprep spin Miniprep KIT (Qiagen). In addition, the nucleotide sequence thereof was determined using Big Dye Terminator v3.1 cycle sequencing kit (ABI) and ABI310 sequencer to confirm that the nucleotide sequence of the mutation-introduced VH was linked to the nucleotide sequence of PE38 in the pRSET vector.

[Introduction of Cysteine Mutation into Immunoglobulin Light Chain Gene Variable Region]

Primers were prepared which were designed so that the 125th amino acid of the immunoglobulin light chain gene variable region (VL) of the mouse anti-rat FR-β monoclonal antibody 4A67 was mutated to cysteine (nucleotide sequence: tgt).

Sense: taa gaa gga gat ata cat atg CAA ATT GTT CTC ACC CAG TCT, SEQ ID NO: 7 (This primer contains the bases catatg cleavable by a restriction enzyme, NdeI; thus, cloning at the site enables the expression of a protein using atg as a start codon.)

Antisense: get ttg tta gca gcc gaa ttc cta TTT TAT TTC CAA CTT TGT CCC ACA GCC GAA CGT, SEQ ID NO: 8 (This primer is designed so that the 125th amino acid is mutated to cysteine (tgt) with the termination codon tag followed by the bases gaattc cleavable by a restriction enzyme, EcoRI.)

The combination of these primers and pfu DNA polymerase (Stratagene) were used to subject the plasmid pCR2.1-TOPO-4A67VL to PCR. The reaction comprises a 30-cycle PCR at 94° C. for 20 seconds, 55° C. for 30 seconds, and 72° C. for 60 seconds, followed by reaction at 72° C. for 5 minutes. Then, the PCR product was purified; the restriction enzymes NdeI (New England Biolabs) and EcoRI (New England Biolabs) were added to the purified product for reaction; the resultant was subjected to electrophoretic development; and DNA of a desired size was recovered from the gel using QIAquick gel extraction kit (Qiagen). To the recovered DNA was added pRSETPE38 treated with the same restriction enzyme as for the restriction enzyme-treated mutation-introduced VL, and Ligation High (Toyobo) was further used to perform the ligation reaction between VH and pRSETPE38. After the end of the ligation reaction, the gene was introduced into the Escherichia coli TOP10F′ (Invitrogen), and a transformant was selected in an LB medium containing 0.1 mg/ml ampicillin. The plasmid pRSET-VL4A67 in the selected transformant was purified using QIAprep spin Miniprep KIT (Qiagen). In addition, the nucleotide sequence thereof was determined using Big Dye Terminator v3.1 cycle sequencing kit (ABI) and ABI310 sequencer to confirm that the amino acid of the mutation-induced VL was mutated to cysteine and that the termination codon tag was placed.

### [Preparation of Recombinant Protein Inclusion Bodies]

50 ng of the plasmid pRSET-4A67VHPE having the mutation-introduced VH incorporated or the plasmid pRSET-VL4A67 having the mutation-introduced VL incorporated was prepared and transfected into Escherichia coli, BL21(DE3), for protein expression. The selection of the transfected Escherichia coli was performed by culture in an LB medium containing 0.1 mg/ml ampicillin at 37° C. for 15 to 18 hours.

Escherichia coli after the end of selection was cultured under conditions of 1,000 ml of super broth medium and 37° C., and cultured until 1.0 to 1.5 was reached at a visible absorbance of 600 nm. After the culture, IPTG (isopropyl-β-D-thio-galactopyranoside) was added to a final concentration of 1 mM to the medium, which was further cultured at 37° C. for 90 minutes. After the end of culture, Escherichia coli was recovered by centrifugation and then suspended to 200 ml using 50 mM Tris buffer (pH 7.4, containing 20 mM EDTA). After the end of suspension, egg-white lysozyme was added to a final concentration of 0.2 mg/ml thereto, which was then reacted at room temperature for 1 hour to lyse Escherichia coli. After the lysis, centrifugation was performed at 20,000×g to recover the precipitate. The precipitate was further suspended to 200 ml in 50 mM Tris buffer (pH 7.4, containing 2.5% TritonX-100, 0.5 M NaCl, and 20 mM EDTA), to which egg-white lysozyme was then added to a final concentration of 0.2 mg/ml, followed by reaction at room temperature for 1 hour. After the end of reaction, centrifugation was performed at 20,000×g to recover the precipitate. The precipitate was further suspended to 200 ml in 50 mM Tris buffer (pH 7.4, containing 2.5% TritonX-100, 0.5 M NaCl, and 20 mM EDTA), and centrifuged at 20,000×g after thorough mixing to recover the precipitate. The precipitate after repeating the operation 5 times was defined as recombinant immunotoxin inclusion bodies, which was further dissolved in 0.1 M Tris buffer (pH 8.0, containing 6 M guanidine hydrochloride and 1 mM EDTA) for adjustment to a final concentration of 10 mg/ml.

### [Preparation of Recombinant Double-Chain Fv Anti-FR-β Immunotoxin]

4A67-VHPE and 4A67-VL prepared above were mixed, and a recombinant double-chain Fv anti-FR-β immunotoxin was prepared.

First, 0.5 ml of VHPE and 0.25 ml of VL were mixed, to which dithiothreitol (DTT) was then added to a final concentration of 10 mg/ml, followed by reduction treatment at room temperature for 4 hours. After the treatment, the resultant was dissolved in 75 ml of 0.1 M Tris buffer (pH 8.0, containing 0.5 M arginine, 0.9 mM oxidized glutathione, and 2 mM EDTA). This solution was allowed to stand at 10° C. for 40 hours to bind VH to VL. After the end of binding, the solution was concentrated to 5 ml using a centrifugal concentrator with molecular weight cutoff of 10,000 (Centricon 10, Amicon), and further diluted with 50 ml of Tris buffer (pH 7.4, containing 0.1 M urea and 1 mM EDTA). This diluted solution was used as a starting material for the purification of the recombinant immunotoxin.

Then, the starting material was adsorbed to an ion-exchange column, Hi-Trap Q (GE), equilibrated in Tris buffer (pH 7.4, containing 1 mM EDTA) at a flow rate of 30 ml/hour, and then washed with Tris buffer (pH 7.4, containing 1 mM EDTA). After washing, the adsorbed recombinant-type immunotoxin was eluted using Tris buffer (pH 7.4, containing 0.3 M NaCl and 1 mM EDTA). The eluted sample was dialyzed against Tris buffer (pH 7.4, containing 1 mM EDTA) and then further purified using an ion-exchange column, POROS HQ (POROS). Specifically, the dialyzed purified substance was adsorbed thereto at a flow rate of 10 ml/minute and washed with Tris buffer (pH 7.4, containing 1 mM EDTA), and the recombinant-type immunotoxin was eluted by setting a concentration gradient of 0 M to 1.0 M NaCl for the buffer. The final preparation of the purified recombinant-type immunotoxin was performed by TSK300SW (Tosoh) gel filtration chromatography. First, endotoxin in the TSK300SW column was removed by washing with 75% ethanol for disinfection for 48 hours. Then, washing was carried out using distilled water for injection in the Japanese Pharmacopoeia, and the TSK300SW column was then equilibrated with saline in the Japanese Pharmacopoeia. After the end of equilibration, the recombinant-type immunotoxin was administered, and the eluate from the column was collected at a flow rate of 0.25 ml/minute. After the collection, it was treated using a 0.22 μm sterilizing filter, confirmed for purity by SDS-PAGE, and then stored at −80° C.

### [Purity Test by SDS-PAGE]

SDS-PAGE (sodium dodecyl sulfate containing-polyacrylamide gel electrophoresis) used a slab gel of 12% polyacrylamide containing 0.1% sodium dodecyl sulfate (SDS), and the mobile phase used an aqueous solution containing SDS having a final concentration of 0.1%, 130 mM glycine, and 25 mM Tris. Each sample was prepared using 100 mM Tris buffer, pH 6.5, containing SDS at a final concentration of 0.1%, and subjected to boiling treatment for 5 minutes. After the end of boiling, the sample was administered to the slab gel and developed by electrophoresis at a constant current of 30 mA. After the development, the recombinant-type immunotoxin was stained with 0.05% Coomassie brilliant blue R solution (Nacalai Tesque).

### [Measurement of Suppression of Cell Proliferation by Immunotoxin]

Rat FR-β-expressing B300-19 cells were added to a 24-well cell culture plate to 5×10⁴ cells/well, to which the immunotoxin and VHPE were each further added to a final concentration of 0 to 1 μg/ml, followed by culture in a CO₂ incubator at 37° C. Cell proliferation at 24, 48, and 72 hours after the culture was measured using Cell Counting Kit-8 (Reagent for cytotoxicity assay, Dojindo Co., Ltd.). The measurement method used a microplate reader (Thermo) according to the accompanying manual.

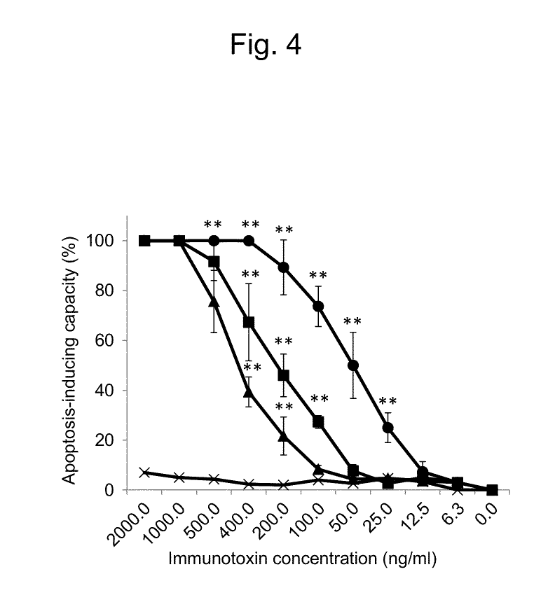

FIG. 4 shows the cell proliferation-suppressing effect (apoptosis-inducing capacity) of the mouse anti-rat FR-β immunotoxin against rat FR-β-expressing B300-19 cells.

In FIG. 4, the vertical axis represents the apoptosis-inducing capacity and the horizontal axis represents the concentration of the mouse anti-rat FR-β immunotoxin (closed triangle, 24 hours; closed square, 48 hours; closed circle, 72 hours) and VHPE (cross, 72 hours) in each culture time. Data indicate the average of the results of 5 independent experiments, and the error bar indicates standard error. **: P<0.01

The immunotoxin suppressed the proliferation of rat FR-β-expressing B300-19 cells depending on the culture time and the addition concentration. In contrast, VHPE used as a control caused no marked suppression of proliferation even under the same conditions. The concentration of the immunotoxin necessary for the 50% suppression of cells (IC50) was 400 ng/ml for 24 hours, 200 ng/ml for 48 hours, and 50 ng/ml for 72 hours.

### Example 3

### Effect of Suppressing Cartilage/Bone Destruction in Methylated Bovine Serum Albumin-Induced Rat Arthritis by Recombinant Immunotoxin

### [Preparation of Methylated Bovine Serum Albumin (Methylated BSA)-Induced Adjuvant Rat Arthritis Model and Administration of Immunotoxin]

The methylated BSA-induced adjuvant rat arthritis model was prepared according to the method of Nicolau Beckmann (Nicolau Beckmann, Magnetic Resonance in Medicine (2003) 49: 1047-1055). Cartilage/bone destruction is known to occur in this arthritis. First, methylated BSA adjusted to 50 μl (5 mg/ml, containing 50% Freund's complete adjuvant) was administered subcutaneously in the abdominal cavity of Lewis rats (female, 6- to 9-week old). In addition, the same operation was carried out 7 days after administration.

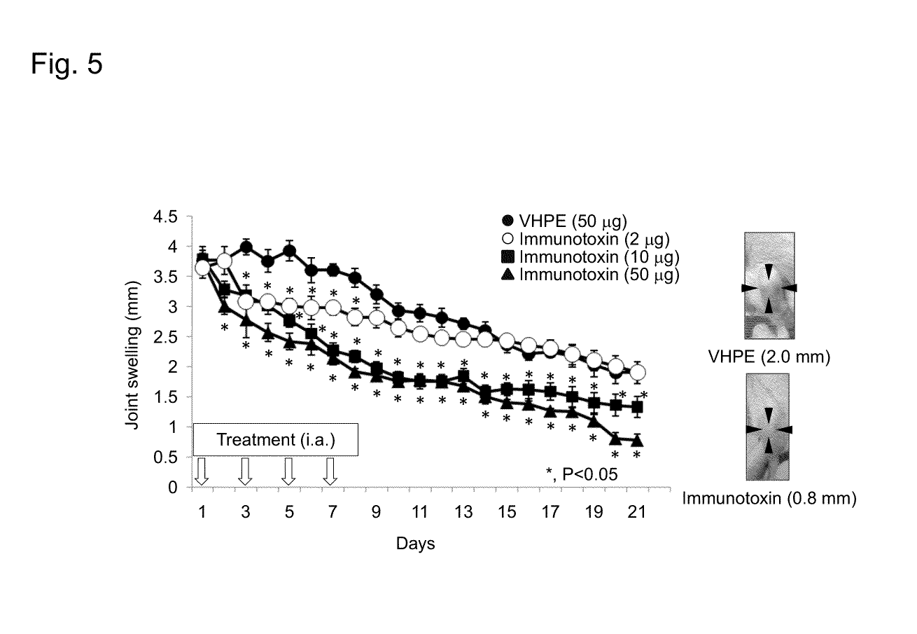

14 days after the 1st subcutaneous administration, methylated BSA adjusted to 50 μl (5 mg/ml PBS) was intra-articularly administered to rats to induce arthritis. The swelling of the joint was confirmed 1 day after the administration of methylated BSA, and the immunotoxin or VHPE as a negative control was then intra-articularly administered. First, a VHPE group (8 rats) or an immunotoxin group (24 rats) was randomly selected, and 50 μg of VHPE adjusted to 50 μl or the immunotoxin (2, 10, or 50 μg) adjusted to 50 μl was administered into the left joint. As a control, saline adjusted to 50 μl was administered into the right joint. The same administration was also performed at the 3rd, 5th, and 7th day after the intra-articular administration of methylated BSA (4 times in total). The swelling of the joint was measured with the passage of days until the 21st day using a caliper. The results of measuring the swelling are shown in FIG. 5.

In FIG. 5, the horizontal axis represents the number of days after inducing arthritis, and the vertical axis represents the width (mm) of the joint increased compared to that of the normal joint. The error bar indicates standard error. *: P<0.05

As shown in FIG. 5, swelling was significantly suppressed in the group of administration of 10 μg or 50 μg of the immunotoxin 3 or more days after the administration of methylated BSA compared to in the VHPE group.

### [Histopathological Analysis and Immunostaining]

The rats were each euthanized at the 21st day after the administration, and both legs were removed and subjected to acetone fixation. After the acetone fixation, the rat joints were subjected to demineralization treatment using 20 mM pH 8.0 Tris buffer containing 0.5 M EDTA. After the demineralization treatment, the tissue was embedded in O.T.C Compound (Sakura) diluted to 50% with pure water. Frozen tissue sections were prepared using an adhesive film according to the method of Kawamoto et al. (Use of a new adhesive film for the preparation of multi-purpose fresh-frozen sections from hard tissues, whole-animals, insects and plants. Arch Histol Cytol. 2003 May; 66(2): 123-43).

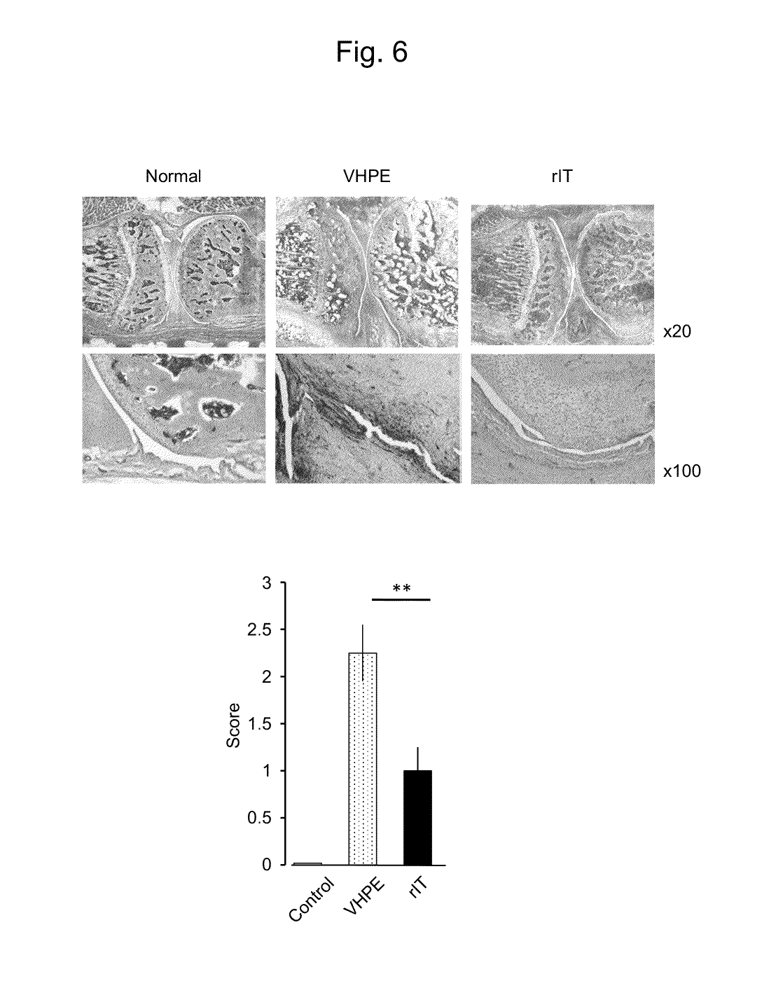

The frozen section was air-dried and then subjected to hematoxylin-eosin staining. After staining, the pathological score of cartilage/bone destruction was analyzed. The pathological score was evaluated in terms of the degree of cartilage/bone destruction with no change; 1 equal to less than 10% lesion; 2 equal to less than 50% lesion; and 3 equal to 50% or more lesion according to the method of Richards P J et al. (Liposomal clodronate eliminates synovial macrophages, reduces inflammation and ameliorates joint destruction in antigen-induced arthritis. Rheumatology (Oxford). 1999 September; 38(9): 818-25), and calculated as grades. FIG. 6 shows the results of histopathological staining in the immunotoxin administration group (rIT) and the VHPE administration group.

The top of FIG. 6 shows the state of each removed joint when subjected to demineralization treatment, thinly sliced, and stained with hematoxylin-eosin. The degree of cartilage/bone destruction was calculated as a grade. The value indicates the average value in each group (8 rats), and the error bar indicates standard error. **: P<0.01

The destruction of cartilage/bone was suppressed in the immunotoxin administration group (rIT) compared to in the VHPE administration group. It turns out that the score of cartilage/bone destruction was also predominantly suppressed in the immunotoxin administration group.

Table 1 shows the comparison of the intra-articular administration of a steroid (methylprednisolone), a hyaluronic acid preparation, and the anti-FR-β immunotoxin of the present invention in the antigen-induced arthritis model.

TABLE 1

| | | | Anti-FR-β |
|---|---|---|---|
| | Steroid¹⁾ | Hyaluronic Acid²⁾ | Immunotoxin |
| Joint Swelling | Ameliorated | Ameliorated but | Ameliorated |
| | | Subsequently | |
| | | Progressed | |
| Histological | No | No | Yes |
| Improvement | | | |
| Improvement of | No | No | Yes |
| Cartilage/Bone | | | |
| Destruction | | | |
| Duration | Short | Short | Long |

¹⁾Green KL, Foong WC. J Pharm Pharmacol. 1993 Sep; 45(9): 815-20.
²⁾Roth A, Mollenhauer J, Wagner A, Fuhrmann R, Straub A, Venbrocks RA, Petrow P, Brauer R, Schubert H, Ozegowski J, Peschel G, Muller PJ, Kinne RW. Arthritis Res Ther. 2005; 7(3): R677-86.

It is reported that the intra-articular administration of steroid rather causes the apoptotic death of cartilage cells (Nakazawa F, Matsuno H, Yudoh K, Watanabe Y, Katayama R, Kimura T. Clin Exp Rheumatol. 2002 November-December; 20(6): 773-81). For the immunotoxin administration, the apoptotic death of cartilage cells is not seen.

Steroid and hyaluronic acid are reported to have no histological improvement effect in a severe osteoarthritis model (Eyigor S, Hepguler S, Sezak M, Oztop F, Capaci K. Clin Exp Rheumatol. 2006 November-December; 24(6): 724). The immunotoxin can be expected to have efficacy against severe osteoarthritis because it histologically improved rheumatoid arthritis with stronger inflammation.

### Example 4

### Potential for Clinical Application

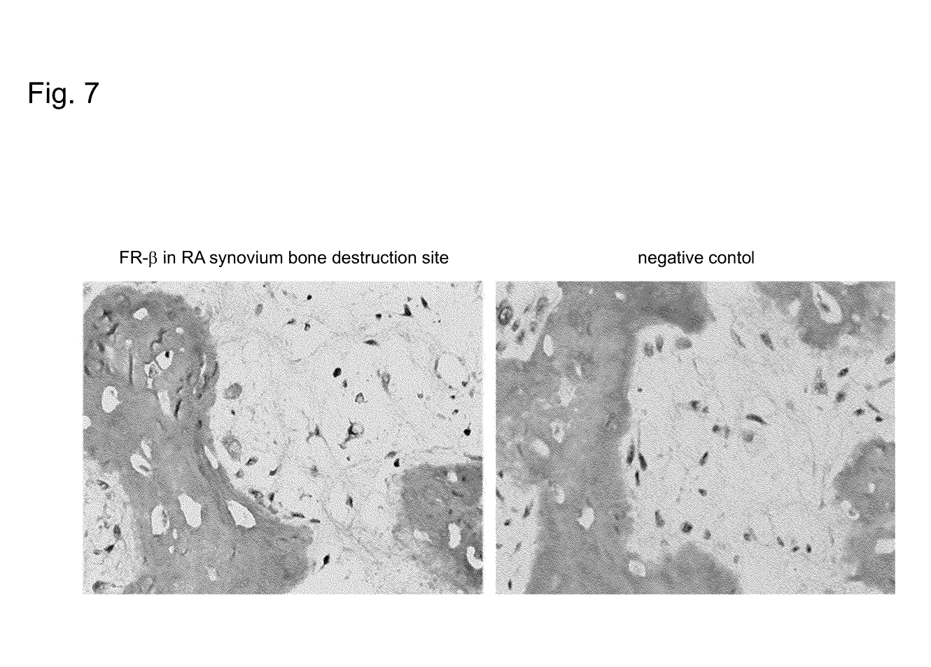

(1) FR-β-expressing cells are present in a bone destruction site in rheumatoid arthritis (see FIG. 7).

Rheumatoid arthritis synovium containing bone was subjected to acetone fixation and then demineralized by substitution in 1% EDTA/phosphate buffer (PBS) with a pH of 7.0 for 2 weeks while daily exchanging the buffer. Thereafter, the tissue was embedded in paraffin, and 5 μm-sections were each attached to a slide for immunostaining. After the treatment at 60° C. for 30 minutes, the section slide was subjected to substitution with xylene for 5 minutes 3 times for deparaffinization treatment and to substitution with ethanol for 5 minutes 3 times and 3-minute substitution with 90% ethanol and with 70% ethanol for dewatering operation.

For antigen recovery, the resultant was autoclaved in Diva Decloaker solution (Biocare Medical, CA, USA) at 120° C. for 10 minutes.

To inactivate endogenous peroxidase, it was reacted in 1% hydrogen peroxide/PBS solution for 10 minutes. 10% goat serum PBS was reacted therewith for 10 minutes to block non-specific adsorption. The resultant was reacted with a mouse anti-human FR-β antibody (94b, IgG1) or a negative control antibody (IgG1) for 30 minutes and washed with PBS 3 times. A peroxidase-labeled goat anti-mouse antibody (Nichirei Biosciences Inc., Tokyo) was reacted therewith for 30 minutes, and the resultant was washed with PBS for 5 minutes 3 times and then color-developed with DAB reagent (Nichirei Biosciences Inc., Tokyo) for 10 minutes. It was washed with PBS 3 times, subjected to hematoxylin staining for 30 seconds, washed with distilled water, dried, and microscopically examined. All reactions were carried out at room temperature. FR-β-expressing macrophages were also observed in the bone destruction site.

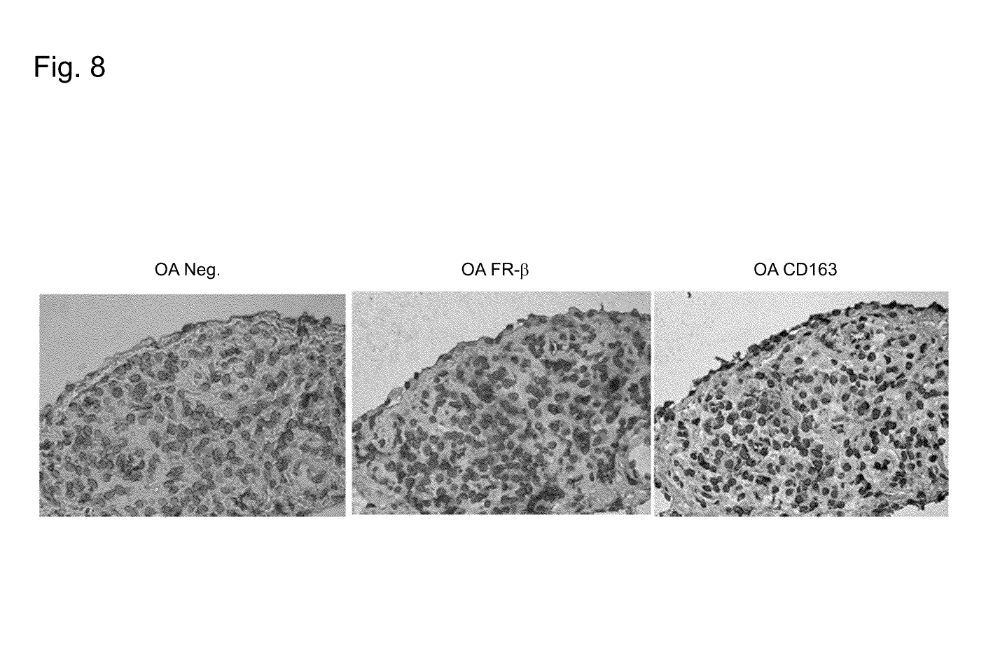

(2) FR-β-expressing cells are present in the osteoarthritis synovium (Tsuneyoshi Y, Tanaka M, Nagai T, Sunahara N, Matsuda T, Sonoda T, Ijiri K, Komiya S, Matsuyama T. Scand J. Rheumatol. 2012; 41(2): 132-40; see FIG. 8).

Osteoarthritis (OA) synovium was subjected to acetone fixation, followed by preparing frozen sections. Therewith was reacted 1% hydrogen peroxide/phosphate buffer (PBS) for 10 minutes to inactivate endogenous peroxidase. The resultant was washed with PBS for 5 minutes 3 times and then reacted with 10% goat serum PBS for 10 minutes to block non-specific adsorption. A mouse anti-human FR-β antibody (94b, IgG1), a mouse anti-CD163 antibody (R20, IgG1), or a negative control antibody (IgG1) was reacted therewith for 30 minutes, followed by washing with PBS 3 times. A peroxidase-labeled goat anti-mouse antibody (Nichirei Biosciences Inc., Tokyo) was reacted therewith for 30 minutes, and the resultant was washed with PBS for 5 minutes 3 times and then color-developed with AEC reagent (Nichirei Biosciences Inc., Tokyo) for 10 minutes. It was washed with PBS 3 times, subjected to hematoxylin staining for 30 seconds, washed with distilled water, dried, and microscopically examined. All reactions were carried out at room temperature. FR-β-expressing macrophages were also observed in the OA synovium.

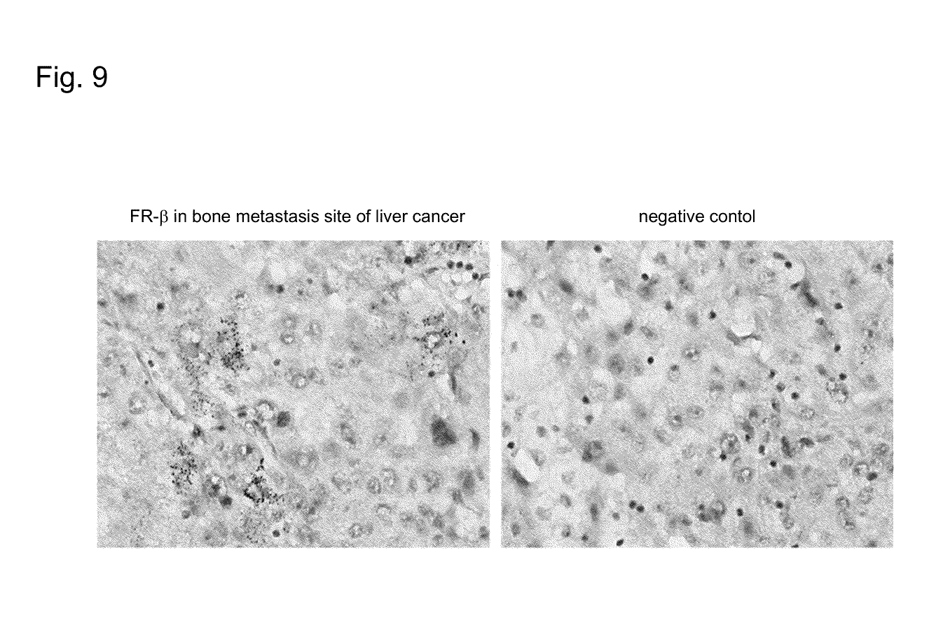

(3) Folate receptor β-expressing cells are present in a site of bone metastasis of liver cancer (see FIG. 9).

The site of bone metastasis of liver cancer was subjected to acetone fixation and then demineralized by substitution in 1% EDTA/phosphate buffer (PBS) with a pH of 7.0 for 2 weeks while daily exchanging the buffer. Thereafter, the tissue was embedded in paraffin, and 5 μm-sections were each attached to a slide for immunostaining. After the treatment at 60° C. for 30 minutes, the section slide was subjected to substitution with xylene for 5 minutes 3 times for deparaffinization treatment and to substitution with ethanol for 5 minutes 3 times and 3-minute substitution with 90% ethanol and with 70% ethanol for dewatering operation.

For antigen recovery, the resultant was autoclaved in Diva Decloaker solution (Biocare Medical, CA, USA) at 120° C. for 10 minutes.

To inactivate endogenous peroxidase, it was reacted in 1% hydrogen peroxide/PBS solution for 10 minutes. 10% goat serum PBS was reacted therewith for 10 minutes to block non-specific adsorption. The resultant was reacted with a mouse anti-human FR-β antibody (94b, IgG1) or a negative control antibody (IgG1) for 30 minutes and washed with PBS 3 times. A peroxidase-labeled goat anti-mouse antibody (Nichirei Biosciences Inc., Tokyo) was reacted therewith for 30 minutes, and the resultant was washed with PBS for 5 minutes 3 times and then color-developed with DAB reagent (Nichirei Biosciences Inc., Tokyo) for 10 minutes. It was washed with PBS 3 times, subjected to hematoxylin staining for 30 seconds, washed with distilled water, dried, and microscopically examined. All reactions were carried out at room temperature. FR-β-expressing macrophages were also observed in the site of bone metastasis.

(4) Detection of Rat Serum Antibody against Toxin of Anti-FR-β Immunotoxin

The intravenous injection of a Pseudomonas exotoxin immunotoxin is reported to result in the high occurrence of a neutralizing antibody against the Pseudomonas exotoxin, which causes the reduction of effect and side effects (Pastan I, Onda M, Weldon J, Fitzgerald D, Kreitman R. Leuk Lymphoma. 2011 June; 52 Suppl 2: 87-90).

Accordingly, the rat serum antibody against the toxin of an anti-FR-β immunotoxin was detected as follows.

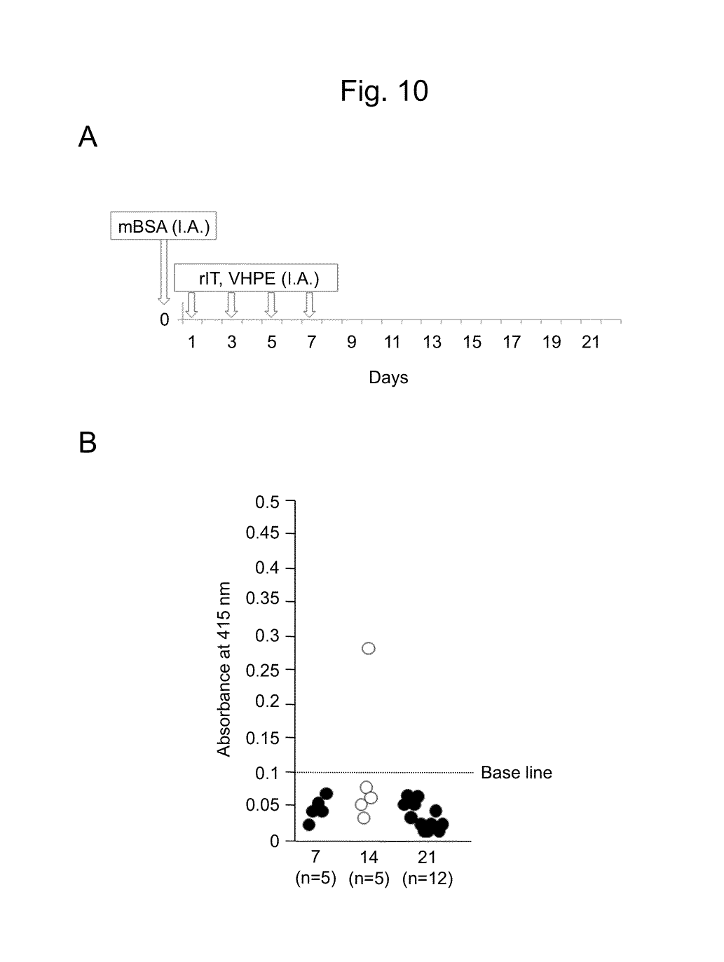

To a methylated BSA-induced rat arthritis model was intra-articularly administered 50 mg of the immunotoxin, and rat serum was collected 7 (n=5), 14 (n=5), and 21 (n=12) days after the induction of arthritis (FIG. 10A).

(Method)

(a) VH-PE38 adjusted to a concentration of 1 μg/ml using 0.1 M carbonate buffer (pH 9.6) was added dropwise to an ELISA plate (MaxiSorp) under a condition of 50 μl (50 ng)/well and incubated at 10° C. overnight. After the end of the incubation, the solution was removed, followed by washing with phosphate buffer (PBS) 3 times. After washing, PBS in which 3% skim milk was dissolved was added dropwise thereto under a condition of 200 μl (50 ng)/well, followed by incubation at 37° C. for 1 hour.

(b) After the end of incubation, a 2-fold dilution series of the rat serum sample was prepared with 3% skim milk PBS and incubated at 37° C. for 1 hour. The positive control used for the anti-immunotoxin antibody was anti-Pseudomonas exotoxin rabbit serum (produced by Sigma). After the end of incubation, the plate was washed with PBS containing 0.1% Tween 20 3 times.

(c) After washing, thereto was dropwise added a horseradish peroxidase-labeled secondary antibody (an anti-rat IgM-IgG produced by SouthernBiotech or an anti-rabbit IgG produced by SouthernBiotech) diluted 1:2,000 with 3% skim milk PBS, under a condition of 50 μl/well, followed by incubation at 37° C. for 30 minutes. After the end of incubation, the plate was washed with PBS containing 0.1% Tween 20 3 times.

(d) After washing, 2,2′-Azino-bis chromogenic substrate solution (produced by Sigma) was added dropwise thereto under a condition of 50 μl/well, followed by incubation at room temperature for 15 minutes. After the end of incubation, absorbance at a wavelength of 415 nm was measured using a plate reader.

(Result)

FIG. 10B shows the reactivity of the rat serum collected 7, 14, or 21 days after inducing arthritis to VH-PE. The value indicates absorbance in each group in 100-fold dilution. All values for the serum of arthritis rats (N=6) not given the immunotoxin were 0.1 or less. One individual in which the absorbance was 0.1 or more was present in the group in which the collection was made 14 days thereafter. All absorbances in the individuals in which the collection was made 7 and 21 days thereafter were 0.1 or less.

Accordingly, the intra-articular administration of the anti-rat FR-β antibody immunotoxin in arthritis rarely results in the occurrence of antibody against Pseudomonas exotoxin, and its intra-articular administration can be expected to be useful.

All publications, patents, and patent applications cited in this specification are intended to be incorporated herein by reference in their entirety.

### INDUSTRIAL APPLICABILITY

According to the present invention, a cartilage/bone destruction suppressor is provided which has a reduced risk of side effects. The cartilage/bone destruction suppressor of the present invention can provide a therapeutic effect against a disease in which cartilage/bone destruction is directly or indirectly observed, by selectively inducing the death or injury of FR-β-expressing macrophages.

### SEQUENCE LISTING FREE TEXT

SEQ ID NO: 1-explanation of artificial sequence: primer
SEQ ID NO: 2-explanation of artificial sequence: primer
SEQ ID NO: 3-explanation of artificial sequence: primer
SEQ ID NO: 4-explanation of artificial sequence: primer
SEQ ID NO: 5-explanation of artificial sequence: primer
SEQ ID NO: 6-explanation of artificial sequence: primer
SEQ ID NO: 7-explanation of artificial sequence: primer
SEQ ID NO: 8-explanation of artificial sequence: primer

1.-5. (canceled)

6. A method for suppressing cartilage destruction comprising administering an effective amount of an antibody against folate receptor β or a complex of the antibody and a biologically or chemically active substance to a subject in need thereof.

7. The method according to claim 6, wherein the antibody against folate receptor β is in the form of a single-chain or a double-chain.

8. The method according to claim 6, wherein the biologically or chemically active substance is at least one selected from toxins, enzymes, cytokines, isotopes, and chemotherapeutic agents.

9. The method according to claim 6, comprising treating a disease characterized by the destruction of cartilage caused by folate receptor β-expressing macrophages.

10. The method according to claim 6, wherein the cartilage destruction is due to rheumatoid arthritis, osteoarthritis, or bone metastasis of malignant tumor.

11. The method according to claim 6, wherein the cartilage destruction is due to osteoarthritis, or bone metastasis of malignant tumor.

12. The method according to claim 6, wherein the cartilage destruction is due to osteoarthritis.

13. A method for suppressing cartilage or bone destruction comprising administering intra-articularly an effective amount of an antibody against folate receptor β or a complex of the antibody and a biologically or chemically active substance to a subject in need thereof.

14. A method for suppressing cartilage or bone destruction comprising administering an effective amount of an antibody against folate receptor β or a complex of the antibody and a biologically or chemically active substance to a subject in need thereof, wherein the cartilage destruction is due to osteoarthritis, or bone metastasis of malignant tumor.
